(19) United States
(12) Patent Application Publication
(10) Pub. No.: US 2021/0272652 A1
(43) Pub. Date: Sep. 2, 2021

(54) Method of finding structural variants for identifying and differentiating species, strains and cells in normal and pathological conditions

(71) Applicant: Xiaoqiu Huang, Ames, IA (US)

(72) Inventor: Xiaoqiu Huang, Ames, IA (US)

(21) Appl. No.: 16/805,783

(22) Filed: Mar. 1, 2020

(51) Int. Cl. G16B 30/10; G16B 20/20

(52) U.S. Cl. CPC G16B 30/10; G16B 20/20

(57) ABSTRACT

Large whole-genome datasets of short reads from species and strains in normal and pathological conditions are processed to find species-, strain- and condition-specific structural variants along with their estimated genome-wide copy numbers. These structural variants provide huge pools of genetic targets with molecular approaches to accurate & fast detection and identification of eukaryotic pathogens such as fungal pathogens and to precise diagnosis and accurate assessment of clinical conditions such as cancer, dementia, Parkinson's disease, Asperger's syndrome.

### CLASSIFICATIONS

C12Q1/6895 Nucleic acid products used in the analysis of nucleic acids, e.g. primers or probes for detection or identification of organisms for plants, fungi or algae

C12Q1/6886 Nucleic acid products used in the analysis of nucleic acids, e.g. primers or probes for diseases caused by alterations of genetic material for cancer

C12Q2600/156 Polymorphic or mutational markers

G16B50/00 ICT programming tools or database systems specially adapted for bioinformatics

G16B30/00 ICT specially adapted for sequence analysis involving nucleotides or amino acids

G16H10/00 ICT specially adapted for the handling or processing of patient-related medical or healthcare data

G16H50/20 ICT specially adapted for medical diagnosis, medical simulation or medical data mining; ICT specially adapted for detecting, monitoring or modelling epidemics or pandemics for computer-aided diagnosis, e.g. based on medical expert systems

G16H10/60 ICT specially adapted for the handling or processing of patient-related medical or healthcare data for patient-specific data, e.g. for electronic patient records

G16H10/40 ICT specially adapted for the handling or processing of patient-related medical or healthcare data for data related to laboratory analysis, e.g. patient specimen analysis

G06F19/00 Digital computing or data processing equipment or methods, specially adapted for specific applications

Y02A90/26 Information and communication technologies [ICT] supporting adaptation to climate change. specially adapted for the handling or processing of medical or healthcare data, relating to climate change for diagnosis or treatment, for medical simulation or for handling medical devices

### DESCRIPTION

An Amendment Directing a Sequence Listing into the Application

In response to the notice to disclose a Sequence Listing, the Sequence Listing is submitted via EFS-Web as an ASCII text file with the application. The applicant hereby states that the Sequence Listing includes no new matter. Every sequence in the Sequence Listing is one of the sequence fragments in Tables 1 and 2 in the previously-submitted specification (see new subsections named “Sequence Listing: fungal discriminating fragments” and “Sequence Listing: human discriminating fragments”). This statement indicates support for the amendment in the application. Because the Sequence Listing is submitted via EFS-Web as an ASCII text file, a substitute specification is provided, consisting of a marked-up version and a clean version. The applicant hereby states that the substitute specification contains no new matter.

### REFERENCE TO THE SEQUENCE LISTING AS AN ASCII TEXT FILE

The content of the ASCII text file of the Sequence Listing named “seq.list.ST.25.txt” with a size of 22,192,697 in bytes and with a creation date of Apr. 23, 2020 is incorporated herein by reference in its entirety.

### CROSS-REFERENCE TO RELATED APPLICATIONS

The present application is not related to previous US patent applications.

This invention was made without government support.

### FIELD OF THE INVENTION

The present invention relates to a data mining method of finding species-, strain-, and condition-specific structural variants by processing whole-genome datasets of short reads from eukaryotic species, strains and cells in normal and pathological conditions. These structural variants can be used as genetic targets with molecular approaches like PCR and hybridizations for the detection and identification of eukaryotic pathogens and as genetic markers and signatures for the diagnosis and assessment of clinical conditions.

### BACKGROUND OF THE INVENTION

Structural variants are differences involving a DNA segment of >50 bp between individuals, or (in cancer) between cells in normal and pathological conditions (Cameron et al. 2019). It is well known that many structural variants are associated with genetic diseases. Finding structural variants is important in understanding human diversity and disease susceptibility. However, structural variants are more difficult to find than single nucleotide polymorphisms (SNPs). All existing methods for finding structural variants in whole-genome datasets of short reads require a reference genome assembly to provide common genomic locations for mapping short reads (Cameron et al. 2019; U.S. Pat. No. 9,721,062). Therefore, these methods cannot find structural variants that are not connected to the reference genome. And it is much more expensive to find structural variants by producing whole-genome datasets of long reads. Thus, it is useful in health care to invent a novel method of finding structural variants in whole-genome datasets of short reads without using a reference genome assembly.

Filamentous fungi include the most important fungal pathogens of plants and animals. For example, some Aspergillus species cause serious disease in humans and animals, and some Fusarium species cause severe crop losses worldwide. Fusarium is a large genus of complex taxonomy with up to 1,000 species identified at times (Nelson et al. 1994). Aspergillus is a genus with a few hundred mold species. Fungal species and strain identification is necessary for effective control and treatment of diseases of plants and animals. Molecular typing schemes that use standard genomic regions have emerged as promising methods for identifying fungal species. These standard genomic regions include the nuclear ribosomal RNA internal transcribed spacer (ITS) region for barcoding the fungal kingdom (Schoch et al. 2012), the translation elongation factor 1-alpha locus for identifying Fusarium (Geiser et al. 2004), and the beta-tubulin or calmodulin locus for identifying Aspergillus (Geiser et al. 2007). However, in many cases, the ITS region is not sufficiently variable to discriminate between some fungi at the species level, and species level identification using single-copy nuclear genes (e.g. beta-tubulin for Aspergillus) is time-consuming and complex in clinical practice, although the identification is clinically necessary in cases (e.g. Aspergillus fumigatus species complex) where differences exist in antifungal susceptibility and clinical outcomes between closely related species (Lamoth 2016; Wickes & Wiederhold 2018). DNA-based tests for the detection and identification of fungal pathogens all use specific DNA sequences (for example, U.S. Pat. No. 6,387,652) or known genes (for example, U.S. Pat. No. 8,114,601) as genetic targets. These DNA sequences or known genes are not sufficiently variable to discriminate between some fungi at the species level.

In filamentous fungi, vegetatively growing cells distinguish self from nonself (allorecognition). During the vegetative growth phase, allorecognition can result in vegetative or heterokaryon incompatibility (HET) following fusion of genetically different cells, which disrupts growth and causes cell death (Glass & Dementhon 2006; Paoletti & Saupe 2009). However, heterokaryon incompatibility is suppressed following conidial anastomosis tube (CAT) fusion between vegetatively incompatible strains of Colletotrichum lindemuthianum (Ishikawa et al. 2012), suggesting that horizontal gene transfer (HGT) may result from CAT fusion (Manners & He 2011). Heterokaryon incompatibility involves a protein partner with a HET domain as a trigger of programmed cell death (Paoletti & Clave 2007). HGT allows for the species- and strain-specific acquisition of certain pathogenicity genes and transposons in multiple highly similar copies, called repetitive elements (Huang 2019). Such an element can be present in a much higher copy number in a particular species than in all other species. Or its variants can be present in high copy numbers in closely related species, and one variant with a sufficiently long unique region can be specific to a particular species. Fungal pathogens such as A. fumigatus (Fedorova et al. 2008) and F. oxysporum (Huang 2019) contain chromosomes with highly similar subtelomeres and with highly similar copies of transposons. These genomic regions are often species-specific and serve as anchors for homologous recombination (Huang 2019). Although the ITS regions of ribosomal DNA (rDNA) may not be sufficiently different between closely related species, other regions (such as intergenic spacers, or IGS) of rDNA may contain species-specific DNA segments, and so are parts of mitochondrial DNA (mtDNA). A sufficiently long element in a high copy number in a particular species can be used as a genomic marker for identification of the species. Such an element is easy to amplify by PCR because it is present in a high copy number. This type of element is useful in building an affordable and accurate multilocus barcoding chip for identification of the species.

There are two main approaches to finding specie-specific sequences from which diagnostic PCR primers and oligonucleotide probes are designed. The first approach is based on a known region of the genome. In this approach, the sequences of a target species and all its closely related neighbor species over the known region are generated and compared to find sequences specific to the target species, for example, the IGS region of F. virguliforme (Wang et al. 2015). This approach is time-consuming and labor-intensive. The second approach selects species-specific sequences over the whole genome. It works with assembled genome sequences of a target species and all its closely related neighbor species. However, long repetitive elements in multiple highly similar copies are missed by genome assembly programs in the reconstruction of genome sequences (Wu et al. 2009). Thus, primer design programs such as RUCS (Thomsen et al. 2017), which work only with assembled genome sequences of closely related species, cannot select candidate primers in elements absent from a positive (target) genome dataset, and cannot remove candidate primers matching elements absent from a negative (non-target) genome dataset. Some of these elements can be recovered by long read sequencing such as Single Molecule, Real-Time (SMRT) from PacBio, which is much more expensive than short read sequencing such as Sequencing By Synthesis (SBS) from Illumina. However, about 10% of 9331 complete bacterial genomes contain complex regions that could not be assembled with SMRT long reads (Schmid et al. 2018). On the other hand, these complex regions can still be present in short reads, which are more accurate than long reads (Schmid et al. 2018). Although repetitive elements cannot be assembled together with unique regions in a genome assembly, specialized method are designed to produce repetitive elements alone from datasets of short reads (Novak et al. 2010; Koch et al. 2014; Goubert et al. 2015). Still, no methods are known to use datasets of short reads to produce sequences and structures for identifying closely related fungal pathogens; these sequences and structures are later used to design PCR primers and hybridization probes for differentiating each fungal pathogen from its close related relatives.

Alignment-free methods have been designed to select diagnostic primers and probes for discriminating viruses and bacteria (Hysom et al. 2012; Pritchard et al. 2012) and to find discriminating k-mers for classing metagenomic sequences (Ounit et al. 2015; Ounit & Lonardi 2016). However, because these methods find diagnostic probes or discriminating k-mers only in assembled genome sequences, they cannot find primers and probes from genome regions that are present in the short reads but are not reconstructed and missing in the assembled sequence, due to short read length, repetitive regions or polymorphisms. And copy number information is missing from the genome assembly. This typing strategy is limited by the completeness of the genome assemblies, because it can neither find specific probes missing in all positive genome assemblies, nor reject non-specific probes missing in all negative genome assemblies. We collectively call these approaches assembly-based methods.

Some genomic regions that are difficult to assemble are missing in the genome assembly. Multiple copies of a repetitive element are assembled into a single copy in the genome assembly. So the number of occurrences of a fragment in a dataset of short reads can differ by a great deal from the number of occurrences of the fragment in an assembled genome. We present results to show that a significant portion of the genome is not represented in the genome assembly.

### SUMMARY OF THE INVENTION

The present invention overcomes the aforesaid deficiencies in the prior art by providing a method of finding structural variants for identifying closely related pathogen species or isolates and for differentiating normal cells from cancer cells or cells harboring disease mutations. Structural variants from the method are in the form of high-copy fragments that are specific to species or isolates, or differentiate between normal cells and cells harboring disease mutations. Because high-copy fragments are hard to assemble, they are underrepresented in genome assemblies. Thus, the new method finds those high-copy fragments in whole-genome datasets of short reads from a number of isolates, without using any genome sequences or assemblies. Note that pathogens like plant fungal pathogens contain host-specific subtelomere sequences that are present in high copy number (Huang 2019) and that human cancer cells harbor ribosomal DNA copy number amplification and loss (Wang & Lemos 2017). Structural variants from the method can be used in the design of a CRISPR-based system as well as PCR and hybridization systems to rapidly determine these variants are present in a biological sample. Such tests are useful in detecting pathogens or genetic disorders.

### BRIEF DESCRIPTION OF THE DRAWINGS

FIG. 1 shows a flowchart illustrating an embodiment of a method of finding structural variants for identifying and differentiating two or more isolates (or types of cells).

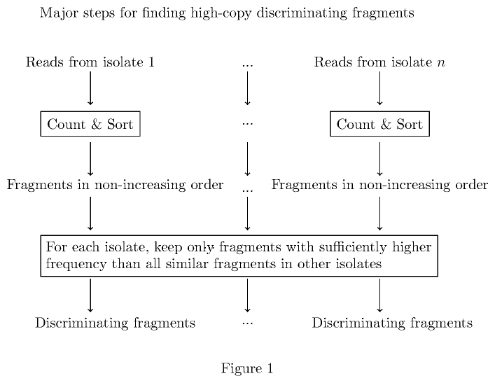

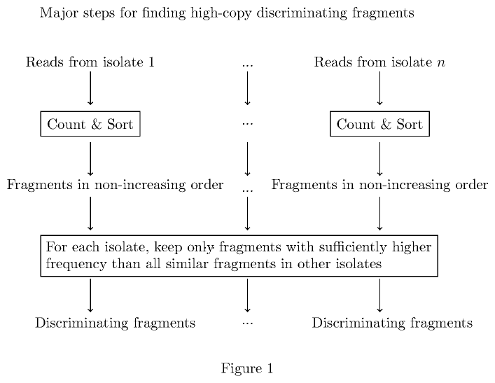

### DETAILED DESCRIPTION

The embodiments presented in this document are illustrative and are not meant to limit the invention. Other embodiments can be constructed without departing from the scope of the claims of the present invention.

We describe a computational method for finding unique high-copy fragments as candidates for PCR primers and oligonucleotide probes in identification and detection of species and isolates, and in differentiation of normal cells from cells harboring disease mutations. In the rest of this document, the term isolate is used to mean what were isolated from the sample in identification of pathogens, and to mean a type of cells in differentiation of normal cells from cells harboring disease mutations. For a number of isolates, with each isolate associated with its datasets of short reads, our method constructs isolate-specific DNA fragments from their datasets of short reads, instead of their assembled genome sequences. The method consists of two major steps, as described below.

Step 1: For each isolate represented by datasets of short reads, build an ordered list of all distinct fragments of length k (also called k-mers) that are contained in any of the short reads in forward or reverse orientation, along with the frequency of each fragment: the number of times it is contained (occurs) in the short reads in forward and reverse orientation. The fragments are listed in a non-increasing order of their frequencies, with the most frequent fragments at the beginning of the list.

Step 2: Produce isolate-specific lists of frequent fragments by keeping, in each isolate, only those fragments with high frequency and with no matches to fragments in other isolates and those fragments with sufficiently higher frequency than all similar fragments in other isolates (the default option), or only those fragments with high frequency and with no matches to fragments in other isolates (an alternative option). Specifically, if the default option is selected, then for frequency count cutoff c and factor cutoff f, the list of fragments for each isolate is revised to keep only those fragments such that if they have no matches to fragments in the other isolates, then their frequency counts are greater than f*c, and if they have matches to fragments in some other isolates, then their frequency counts are f times greater than those of the matched fragments in the other isolates. Through the alternative option, the resulting list contains only those fragments that have their frequency counts greater than f*c and that have no matches to fragments in other isolates. Those fragments are said to be unique to the isolate with respect to the other isolates. The frequency count cutoff c is used to exclude the fragments with frequency counts less than c, which are considered to contain errors. In one embodiment, a fragment g from one list and a fragment h from another list are said to be similar (have a match) if there is a gap-less match of length s (allowing for base mismatches) between the fragments g and h. The match length s can be set to a value between k/2 and k, where k is the fragment length.

Implementation

We first describe a method for constructing a general superword array (Huang et al. 2006; Huang 2017). Then we show how to implement steps 1 and 2 by building particular superword arrays.

A DNA word of length w is a string of w bases, with each base being is one of four regular bases A, C, G and T, or a non-regular base like N. A word model is represented by a sequence of (one or more) 1's and (zero or more) 0's, with each 1 bit denoting a checked position and each 0 bit denoting an unchecked position (Ma et al. 2002). In one embodiment, consider a word model of length 15 with 12 checked positions and 3 unchecked positions: 110110111110111. Two DNA words of length 15 form a match if they have the same regular base at each of the checked positions, regardless of whether they have base mismatches or non-regular bases at the unchecked positions. For example, the two 15-bp DNA words below on the left form a match, but those in the middle or on the right do not form a match.

| | | |
|---|---|---|
| 110110111110111 | 110110111110111 | 110110111110111 |
| ACGTGTCACGANCGA | TCGTGTCACGANCGA | NCGTGTCACGANCGA |
| ACATGCCACGATCGA | ACATGCCACGATCGA | ACATGCCACGATCGA |
| A match | Not a match | Not a match |

Below we define the code of a word under a word model of w bits so that two DNA words of length w form a match if and only if they have the same non-negative code. Let w be the length of the word model with e checked positions and u unchecked positions such that w≥e≥1. Then w=e+u. To calculate the code of a word of length w under the word model, the word is transformed into a string of e bases a₁a₂ . . . a_{c} by removing the bases at each unchecked position according to the word model. If any of these remaining bases is a non-regular base, then the code of the word is −1. Otherwise, the code of the word is defined to be the code of the resulting string:

code(a₁a₂. . . a_{d})=d(a₁)×4^{d-1}+d(a₂)×4^{e-2}+ . . . +d(aₑ)×4⁰, (1)

where d(A) is 0, d(C) is 1, d(G) is 2 and d(T) is 3.

A superword with v words of length w is a string of v*w characters, obtained by concatenating the v words in order (Huang et al. 2006; Huang 2017). The v word positions of the superword from left to right are referred to as word positions 1 through v. For example, the word of the superword at word position v refers to the rightmost word of the superword.

The value for the parameter v is selected such that the superword length v*w (also called the minimum overlap length) is smaller than the length r of each read. For example, for reads of length 100, we can set w to 15, and v to 6, resulting in a superword length of 90. A read of length r in forward orientation has r−v*w+1 superwords, starting at positions 1, 2, . . . , r−v*w+1, and in reverse orientation has another r−v*w+1 superwords. Two superwords form a match or are identical if they have identical regular bases at each checked word position. One superword is less (greater) than another superword if the string of bases at each checked position of the first superword, in lexicographic order, comes before (after) the string of bases at each checked position of the second superword. Each read is given a unique nonnegative integer, called a read index. Then each superword in each read in each orientation can be given a unique nonnegative integer, called a superword index, computed from the orientation of the read, the start position of the superword in the read in the orientation and the read index. There is also an inverse function that is efficiently used to produce, from a superword index, the orientation of the read, the start position of the superword in the read in the orientation, and the read index. The superword array for a set of reads is an array of all superword indexes that are sorted in the lexicographic order of the superwords.

Below is an example of eight superwords in a sorted order. Each superword is made up of four words of length 15, where each checked position of the word is indicated by the bit 1, and each unchecked position by the bit 0. The last position of each word in the superword is marked by a pound sign. The top three superwords are considered identical because they have identical bases at each checked position. The middle two superwords are also identical, and so are the bottom three superwords. Superwords in each block may have different bases or the undetermined base N at an unchecked position. The top block comes before the middle block because the top block has the base G at a checked position marked by an asterisk and the middle block has the base T at the same position. Likewise, the middle block comes before the bottom block, as determined by the bases at another checked position marked by an asterisk.

```
110110111110111110110111110111110110111110111110110111110111
                            # # # #
ATNAGCCCAGTTATCCTAGTCAGACTCAGGTTNCATCATTCNTCCGANCAGACTGACCAG
ATGAGGCCAGTCATCCTTGTGAGACTTAGGTTACATCATTCATCCGAACAAACTGANCAG
ATTAGNCCAGTAATCCTCGTAAGACTAAGGTTACANCATTCTTCCGACCANACTGATCAG
                                    *
ATCAGTCCAGTNATCCTTTTTAGACTTAGGTTGCAACATTCGTCCGAGCATACTGACCAG
ATGAGACCAGTNATCCTATTGAGACTGAGGTTACATCATTCTTCCGACCAAACTGAACAG
                                                                                *
ATGAGACCAGTNATCCTNTTNAGACTCAGGTTCCAACATTGCTCCGANCATACTGAGCAG
ATNAGTCCAGTAATCCTTTTAAGACTTAGGTTGCAGCATTGGTCCGAGCANACTGACCAG
ATCAGNCCAGTNATCCTATTTAGACTNAGGTTGCATCATTGATCCGATCACACTGANCAG
```

The superword array is constructed in v rounds of sorting. First the array is initialized to the superword indexes in an increasing order of their values. Then for each word position p from v to 1 (from right to left in the superword), the array is sorted in the lexicographic order of words of every superword at word position p. The sorting is performed by using a lookup table along with a location array as buckets for all strings of length c, where the location array is an integer array with its index running from 0 to the largest superword index. The lookup table is initially set to a negative value (denoting the empty bucket) at each index, the code of each string of length c. The bucket sorting is done by placing the elements of the superword array from left to right into their buckets and then by copying the elements in the buckets in reverse order back to the superword array from right to left. Specifically, the current element, a superword index, of the superword array is placed into its bucket as follows. The current superword index is used to find its word at word position p. Then the word of length w is turned into a string of length c by using bitwise operations to remove bases at every unchecked position. Next the code of the resulting string is used as an index into the lookup table, the lookup table value at the index is saved in the location array at the superword index, and the lookup table at the index is set to the superword index. After all the elements of the superword array are entered into the buckets constructed with the lookup table and the location array, the elements in the buckets, in reverse order starting with the largest bucket, are copied back to the superword array from right to left.

After its construction, the superword array is partitioned into sections with each section composed of identical superwords, which are found one by one by using a binary search.

In one embodiment, step 1 is implemented by constructing, for each isolate, a superword array for its dataset of short reads. For short reads of at least 100 bp, all superwords are of length 96=8*12, where each superword consists of 8 words of length 12. The word model is the sequence of 12 1's with no 0's. So each block of identical superwords in the superword array corresponds to all occurrences of a distinct fragment of 96 bp. The size of the block is the frequency of the superword (a distinct fragment of 96 bp) in the short reads in forward and reverse orientation. The fragments are arranged in a non-increasing order by their frequencies. Now each isolate is represented by its ordered list of distinct fragments of length k with each fragment associated with its frequency (≥1).

Step 2 is implemented by constructing a superword array for all files of fragments, one file of fragments per isolate with each fragment treated as a read. In one embodiment, for fragments of 96 bp, the following word model of length 24 is used 111011010110001100100111

The number v of words in every superword is set to 2 so that the length of all superwords is 48. Then each block of identical superwords in the superword array corresponds to matches of 48-bp superwords of some 96-bp fragments. For each block of identical superwords, if the block contains superwords of 96-bp fragments from two or more files (two or more isolates), then these fragments from two or more isolates have 48-bp superword matches (allowing for base mismatches). The fragments in the block are removed from their files unless there is a fragment whose frequency is f times higher than the larger of c and the frequencies of all fragments from other isolates. If there is such a fragment, the fragment is kept in its file. Note that for a block with all fragments from the same file, the fragments are removed from the file unless there is a fragment whose frequency is higher than f×c. So at the end of this process, only fragments whose frequencies are f times higher than the larger of c and the frequencies of all similar fragments in other isolates remain in their files.

We implemented the above method as programs Freq for step 1, Diff for step 2 through the default option, and Uniq for step 2 through the alternative option. Freq takes as input files of short reads from an isolate and produces as output a file of distinct fragments of length k each with its frequency in the short reads in forward and reverse orientation. The fragments in the file are arranged in a non-increasing order by their frequencies, with the most frequent fragments placed at the beginning of the file. Diff and Uniq take as input a number of files of fragments, with each produced by Freq on an isolate, and produces as output the same number of files of isolate-specific fragments. Each output file of isolate-specific fragments from the Uniq program contains those fragments in the corresponding input file that have high frequency but have no gap-less matches of length s (allowing for base mismatches) to fragments in the other input files. Each output file from the Diff program contains those fragments from Uniq plus additional fragments with sufficiently higher frequencies that the frequencies of all similar fragments in the other input files. The fragments in each output file are still ordered by their frequencies with the most frequent fragments at the beginning of the file.

Finding Structural Variants to Identify Closely Related Species

We present an application of the method in one embodiment. To show that it is better to mine discriminating fragments in short reads than in assembled genomes, we selected A. fumigatus and four other pathogenic species in Aspergillus section Fumigati, and Fusarium virguliforme and three other pathogenic species in the Fusarium solani species complex. The five Aspergillus species have little variation in their ITS region, and so are the four Fusarium species. For each of the nine species, a dataset of short reads for an isolate from the species was identified by its accession number from Sequence Read Archive (SRA) at The National Center for Biotechnology Information (NCBI).

We ran Freq on each dataset of short reads to produce a file of distinct 96-bp fragments each with its occurrence count. Then we ran Uniq (with the match length s set to 48) on the five files of 96-bp fragments for the Aspergillus isolates to produce five files of unique fragments. For each file of unique 96-bp fragments, top 50,000 (most frequent) fragments were taken from the file and were compared by Blastn (Zhang et al. 2000) to the database of all assembled genomes (including the assembled genome of the isolate itself) in the Aspergillus genus. We also repeated this process on the four files of 96-bp fragments for the Fusarium isolates, with the database of all assembled genomes (including the assembled genome of the isolate itself) in the Fusarium genus. Table 1 shows the species of each isolate, the accession number of each dataset of short reads, and occurrence count ranges for top 50,000 fragments, and the number and percentage of those fragments that have no matches to the database of the assembled genomes.

We found that 8,312 (or about 16.6%) of the 96-bp fragments each occurring 69 to 2,603 times in the short reads (NCBI SRA accession SRR3757108) of A. novo-fumigatus IBT 16806 have no match of ≥77% percent identity to 191 assembled genomes (including the assembled genome of this isolate) in the Aspergillus genus. This finding and similar results for four other isolates in Aspergillus section Fumigati are shown in Table 1. Also reported in Table 1 are results for several closely related isolates in the Fusarium solani complex: for each isolate, the number and percentage of 96-bp fragments with no matches to 252 assembled genomes (including (including the assembled genome of this isolate) in the Fusarium genus.

The results show that a significant portion of frequent fragments are present in the short reads but have no matches to the assembled genomes. Thus, there are two problems with mining discriminating fragments in assembled genomes. One problem is that discriminating fragments that are present only in an isolate but are missing in

TABLE 1
Number and percentage of 96-bp fragments (in each of several isolates)
with no matches to all assembled genomes (including its own) in the genus

| | NCBI SRA | Fragment | No. (%) of fragments |
|---|---|---|---|
| Isolate | accession | count range | with no matches |
| A. thermomutatus HMR AF 39 | SRR8165488 | 82-4038 | 10410 (20.1%) |
| A. novofumigatus IBT 16806 | SRR3757108 | 69-2603 | 8360 (16.7%) |
| A. viridinutans FRR 0576 | SRR9026512 | 29-6744 | 5241 (10.5%) |
| A. fumigatus JCM 10253 | DRR032500 | 110-5460 | 3149 (6.3%) |
| A. turcosus HMR AF 23 | SRR8165489 | 82-4945 | 940 (1.9%) |
| F. azukicola NRRL 54364 | SRR2102277 | 98-9999 | 8905 (17.8%) |
| F. cuneirostrum NRRL 31157 | SRR2098566 | 28-1113 | 5799 (11.6%) |
| F. phaseoli NRRL 31156 | SRR2098571 | 27-1932 | 2839 (5.7%) |
| F. virguliforme NRRL 34551 | SRR2098563 | 49-1285 | 2044 (4.1%) |

its assembled genome could not be selected by assembly-based approaches. The other problem is that non-discriminating fragments that are present in two isolates but are missing only in one of the two assembled genomes are selected as discriminating ones by assembly-based approaches.

We also used the new method to find discriminating fragments for isolates within the species of F. virguliforme. Note that F. virguliforme is a young pathogenic species with low single nucleotide polymorphism (SNP) rates less than 1 in 50 kb in core chromosomes between isolates within this species (Huang et al. 2016). We selected two databases of short reads (NCBI SRA accession SRR2098554) for isolate F. virguliforme LL0009 and (NCBI SRA accession SRR2096988) for isolate F. virguliforme Clinton-1B. We ran Freq on each dataset of short reads to produce a file of distinct 96-bp fragments. Then we ran Uniq on the two files of fragments to produce two files of unique fragments, where unique fragments in one output file had no strong matches (with percent identity above 93%) to ones in the other output file. We selected top 25,000 fragments with an occurrence count range of 35 to 1103 from the file of unique fragments for isolate LL0009, and compared them by Blastn with each of the assembled genomes for isolates LL0009, Clinton-1B, NRRL 34551, and Mont-1, all from the species of F. virguliforme. Of the 25,000 fragments, 3287 (13.1%) had no matches to the genome assembly of isolate LL0009, and 19,413 to 22,974 (71.9% to 79.1%) had no matches of ≥77% percent identity to the genome assemblies of the other three isolates. In particular, 168 fragments with occurrence counts of 822 to 1103 had no matches to the genome assemblies of the other three isolates, indicating that these high-copy fragments discriminate LL0009 from the other three isolates in this species.

Sequence Listing: Fungal Discriminating Fragments

The fungal discriminating fragments in Table 1 are provided in part 1 of the Sequence Listing, using the symbols and format in accordance with the requirements of 37 CFR 1.821-1.825. The sequences of these fragments (SEQ ID NO: 1 to SEQ ID NO: 47687) are listed in the order of their species in Table 1. The occurrence count of each fragment is given as an integer after numeric identifier <223> in the <220> to <223> feature section. For each species, the sequences of its fragments are listed in a non-increasing order of occurrence counts.

Finding Structural Variants to Distinguish Normal Cells from Cancer Cells

We present an application of the method in another embodiment. For frequency count and factor cutoffs c and f, let SV_{c,f}(U|S, T) denote structural variants in the form of fragments whose frequency counts in isolate U are f times higher than the larger of c and the frequency counts of all similar fragments in isolates S or T. The structural variants in SV_{c,f}(U|S,T) are said to be unique to isolate U with respect to isolates S and T. Below we give a precise definition of the notation SV_{c,f} (U|S,T). For a fragment u in isolate U, let freq(u) denote its frequency in the dataset of reads from U. The frequency count cutoff c is used to exclude the fragments with frequency counts less than c, which are considered to contain errors. A fragment u from isolate U is a structural variant in SV_{c,f} (U|S,T) if freq(u)≥f×freq(t) and freq(t)≥c hold for every fragment t in isolates S or T with a superword match to fragment u, or if freq(u)≥f×c holds in case fragment u has no superword matches to any fragments in isolates S or T with frequency counts equal to or higher than c.

We illustrate the use of the new method by providing results from its implementation on datasets of reads from four female human cell lines: healthy B lymphocyte cell line NA12878 (NCBI SRA accession SRR9644381), healthy B lymphocyte cell line NA24695 (SRR2831468), ovary cancer cell line SNU-251 (SRR10418660), and gastric cancer cell line SGC-7901 (SRR10447757). Below we use single-letter names for these four cell lines: W (White) for NA12878, C (Chinese) for NA24695, 0 (Ovary) for SNU-251, and G (Gastric) for SGC-7901. The dataset from each of the four cell lines consists of a pair of files storing paired-end reads of 150 bp. For each cell line, only the first of its two files, at 12.7-18.1X genome coverage, was used as input to the Freq program. In one embodiment, Freq produced a file of 120-bp fragments each with its frequency count, arranged in a non-increasing order of frequency counts. The Diff took input files of fragments from different cell lines and produced a file of structural variants unique to each of the cell lines with respect to the rest, with the frequency count cutoff set to 20 and frequency factor cutoff set to 10, and the superword model of 116 bits obtained by concatenating 4 copies of the 29-bit word model 11001100110010001100110011011. Note that two 120-bp fragments had a superword match of 116 bp if they had a gapfree alignment of 116 bp with a base match at every 1-bit position of the superword model. Other word models can be used with the new programs.

For each combination of two out of the four human cell lines, Diff produced, without using any genome assembly or database sequences, structural variants unique to one cell line with respect to the other. For example, SV_{20,10}(G|W) contained 421,963 distinct fragments whose frequency counts in cell line G were 10 times higher than the larger of 20 and those of similar fragments in cell line W. The frequency count of the first fragment in this set was 3850. A comparison of the fragment with the GenBank NR/NT nucleotide database revealed that it had perfect matches to genome assemblies of the bacterium Mycoplasma pulmonis, but no matches to any human sequences. A further comparison between the set of fragments and two M. pulmonis genome assemblies found 407,978 fragments having matches to parts of the bacterium genome. For example, the first fragment with an frequency count of 3850 had a total of 23 matches to the two genome assemblies, confirming that the fragment was part of a structural variant in the genome. Note that according to the NCBI SRA accession SRR10447757 record, cell line G was from a study entitled ‘Whole-genome sequencing H. pylori-infected gastric cancer cells’, but our sequence analysis showed that the bacterium pathogen in the cells was M. pulmonis, not H pylori. And because some of the fragments might come from a strain of M. pulmonis whose genome could differ in highly variable antigene regions from the two M. pulmonis genome assemblies, it was likely that SV_{20,10}(G|W) contained M. pulmonis fragments that had no matches to the two M. pulmonis genome assemblies. So we used a genome assembly-free approach to removing these M. pulmonis fragments from SV_{20,10}(G|W). By using a variant of Diff, we found a subset of 472 fragments, out of SV_{20,10}(G|W), with a superword match of 58 bp to the input set of fragments from cell line W, indicating that those 472 fragments might come from human sequences. The highest frequency count of these 472 fragments was 2818. Of these 472 fragments, 283 had matches to parts of human mitochondrion sequences.

Similarly, the output dataset SV_{20,10} (W, C) contained unique fragments from Epstein-Barr Virus.

For six combinations of two human cell lines U and S out of the four, SV_{20,10}(U|S) contained structural variants unique to cell line U with respect to cell line S. These six combinations are shown in column 1 of Table 2. For example, the output datasets SV_{20,10}(W|O) and SV_{20,10}(C|O) contained structural variants with at least 10 times higher frequency counts in the healthy cell lines W and C than in the ovary cancer cell line O. Recall that the dataset FG(O) is a sorted (in a non-increasing order of frequency counts) file of 120-bp fragments each with its frequency count in the dataset of short reads from cell line O. The highest frequency count of fragments in FG(O) having a match to part of a 45S rDNA repeat was 5403, that in FG(W was 6513, and that in FG(C) was 2563. So the number of 45S rDNA repeats in the cancer cell line O was not lower than in the healthy cell line C. However, no fragments in FG(O) with frequency counts equal to or larger than 20 had matches to parts of the 157-bp and 1869-bp DNA sequences encoding for the human 5.8S and 18S RNA genes, respectively. The highest frequency count of fragments in FG(O) having a match to part of the DNA sequence encoding for the human 28S RNA gene was 75. These observations help explain why SV_{20,10}(W|O) and SV_{20,10}(C|O) contained many fragments with high frequency counts from the three RNA gene coding regions, which were unique to cell lines W and C with respect to cell line O, respectively (Table 2). These results suggest that many 45S rDNA repeat units in cell line O were not full-length, missing the 18S, 5.8S and 28S gene regions. Similarly, FG(O) contained few fragments with frequency counts above 20 from human mitochondrion DNA. And Table 2 also shows that cell line G was low in copy number in parts of the region encoding for the 28S RNA gene. Note that our de novo method found structural variants of RNA genes unique to normal cells with respect to cancer cells through an analysis of datasets of short reads from normal and cancer cells, without using any assembled rDNA sequence; a previous study obtained similar results by mapping short reads onto an assembled rDNA sequence (Wang & Lemos, 2017).

Sequence Listing: Human Discriminating Fragments

The human discriminating fragments in Table 2 are provided in part 2 of the Sequence Listing, using the symbols and format in accordance with the requirements of 37 CFR 1.821-1.825. The sequences of these fragments (SEQ ID NO: 47688 to SEQ ID NO: 84193) are listed in the order of their isolate pairs (rows) in Table 2, and are further arranged in the order of their sequence types (columns) for the same row in Table 2. The occurrence count of each fragment is given as an integer followed by its isolate pair and sequence type labels after numeric identifier <223> in the <220> to <223>feature section. For each isolate pair and each sequence type, if the number at this intersection entry of Table 2 is not 0, then the sequences of the fragments corresponding to this entry are listed in a non-increasing order of occurrence counts. For example, SEQ ID NO: 47688 is the sequence of the fragment with the highest occurrence count for isolate pair SV_{20,10}(W|O) and sequence type 18S and its free text is of the form: <223> 990 for SV(W|O) and 18S, where 990 is the occurrence

TABLE 2
Some of the structural variants whose frequency counts in one cell line are
at least 10 times higher than those of similar ones in the other cell line: The number
of fragments (with a range of their frequency counts in parentheses) having a perfect
match to part of an RNA gene or a similar match to part of mitochondrion

| Variants for each | | | | |
|---|---|---|---|---|
| pair of cell lines ^{a} | 18S | 5.8S | 28S | mtDNA |
| SV_{20,10}(W \| O) | 1343 (610-990) | 31 (740-885) | 2616 (206-930) | 11570 (411-1934) |
| SV_{20,10}(C \| O) | 1734 (375-862) | 38 (496-600) | 2512 (206-733) | 15439 (212-1108) |
| SV_{20,10}(W \| G) | 0 | 0 | 204 (293-693) | 237 (411-1934) |
| SV_{20,10}(C \| G) | 0 | 0 | 169 (232-516) | 287 (363-871) |
| SV_{20,10}(W \| C) | 0 | 0 | 10 (301-336) | 148 (426-1738) |
| SV_{20,10}(C \| W) | 0 | 0 | 0 | 168 (229-811) |

^{a} SV_{20,10}(U | S) denotes structural variants whose frequency counts in cell line U are 10 times higher than those of similar ones in cell line S (see text for precise definition). Isolate notations: W (White), NA12878; C (Chinese), NA24695; O (Ovary), SNU-251; G (Gastric), SGC-7901. count, SV(W|O) is the isolate pair label with the subscripts omitted, and 18S is the sequence type. Note that the subscripts in the isolate pair label are difficult to produce in ASCII text.

### REFERENCES

[1] Cameron D L, Stefano L, Papenfuss A T. 2019. Comprehensive evaluation and characterisation of short read general-purpose structural variant calling software. Nature Communications 10: 3240.
[2] Fedorova N D, Khaldi N, Joardar V S, et al. 2008. Genomic islands in the pathogenic filamentous fungus Aspergillus fumigatus. PLoS Genetics 4: e1000046.
[3] Geiser D M, Jimenez-Gasco M D, Kang S C, Makalowska I, Veeraraghavan N, Ward T J, Zhang N, Kuldau G A, O'Donnell K. 2004. FU.S.A.RIUM-ID v. 1.0: A DNA sequence database for identifying Fusarium. European Journal of Plant Pathology 110: 473-479.
[4] Geiser D M, Klich M A, Frisvad J C, Peterson S W, Varga J, Samson R A. 2007. The current status of species recognition and identification in Aspergillus. Studies in Mycology 59: 1-10.
[5] Glass N L, Dementhon K. 2006. Non-self recognition and programmed cell death in filamentous fungi. Current Opinion in Microbiology 9: 553-558.
[6] Goubert C, Modolo L, Vieira C, ValienteMoro C, Mavingui P, Boulesteix M. 2015. De novo assembly and annotation of the Asian tiger mosquito (Aedes albopictus) repeatome with dnaPipeTE from raw genomic reads and comparative analysis with the yellow fever mosquito (Aedes aegypti). Genome biology and evolution 7: 1192-1205.
[7] Hihlal E, Braumann I, van den Berg M, Kempken F. 2011. Suitability of Vader for transposon-mediated mutagenesis in Aspergillus niger. Applied and Environmental Microbiology 77: 2332-2336.
[8] Huang X, Das A, Sahu B B, Srivastava S K, Leandro L F, O'Donnell K, Bhattacharyya M K. 2016. Identification of highly variable supernumerary chromosome segments in an asexual pathogen. PLoS ONE 11: e0158183.
[9] Huang X. 2017. Sequence assembly. In Keith J M. (ed.) Bioinformatics—Volume 1: Data, Sequence Analysis, and Evolution. Humana Press, Second Edition, 35-45.
[10] Huang X. 2019. Host-specific subtelomere: Genomic architecture of pathogen emergence in asexual filamentous fungi. bioRxiv doi: https://doi.org/10.1101/721753.
[11] Huang X, Yang S-P, Chinwalla A, Hillier L, Minx P, Mardis E, Wilson R. 2006. Application of a superword array in genome assembly. Nucleic Acids Research 34: 201-205.
[12] Hysom D A, Naraghi-Arani P, Elsheikh M, Carrillo A C, Williams P L, Gardner S N. 2012. Skip the alignment: degenerate, multiplex primer and probe design using K-mer matching instead of alignments. PLoS ONE 7: e34560. Notes: used genome assemblies. An input file of genome sequences in Fasta format.
[13] Ishikawa F H, Souza E A, Shoji J-Y, Connolly L, Freitag M, Read N D, Roca M G. 2012. Heterokaryon incompatibility is suppressed following conidial anastomosis tube fusion in a fungal plant pathogen. PLoS ONE 7: e31175.
[14] Koch P, Platzer M, Downie B R. 2014. RepARK-de novo creation of repeat libraries from whole-genome NGS reads. Nucleic acids research 42: e80.
[15] Lamoth F. 2016. Aspergillus fumigatus-related species in clinical practice. Frontiers in Microbiology 17: 683.
[16] Ma B, Tromp J, Li M. 2002. PatternHunter: faster and more sensitive homology search. Bioinformatics 18: 440-445.
[17] Manners J M, He C. 2011. Slow-growing heterokaryons as potential intermediates in supernumerary chromosome transfer between biotypes of Colletotrichum gloeosporioides. Mycological Progress 10: 383-388.
[18] Nelson P E, Dignani M C, Anaissie E J. 1994. Taxonomy, biology, and clinical aspects of Fusarium species. Clinical Microbiology Reviews 7: 479-504.
[19] Novak P, Neumann P, Macas J. 2010. Graph-based clustering and characterization of repetitive sequences in next-generation sequencing data. BMC Bioinformatics 11: 378.
[20] Ounit R, Lonardi S. 2016. Higher classification sensitivity of short metagenomic reads with CLARK-S. Bioinformatics 32 3823-3825.
[21] Ounit R, Wanamaker S, Close T J, Lonardi S. 2015. CLARK: fast and accurate classification of metagenomic and genomic sequences using discriminative k-mers. BMC Genomics 16: 236.
[22] Paoletti M, Clave C. 2007. The fungus-specific HET domain mediates programmed cell death in Podospora anserina. Eukaryot Cell 6: 2001-2008.
[23] Paoletti M, Saupe S J. 2009. Fungal incompatibility: evolutionary origin in pathogen defense? BioEssays 31: 1201-1210.
[24] Pritchard L, Holden N J, Bielaszewska M, Karch H, Toth I K. 2012. Alignment-free design of highly discriminatory diagnostic primer sets for Escherichia coli O104:H4 outbreak strains. PLoS ONE 7: e34498. Notes: used genome assemblies.
[25] Schmid M, Frei D, Patrignani A, Schlapbach R, Frey J E, Remus-Emsermann M N P, Ahrens C H. 2018. Pushing the limits of de novo genome assembly for complex prokaryotic genomes harboring very long, near identical repeats. Nucleic Acids Research 46 8953-8965.
[26] Schoch C L, Seifert K A, Huhndorf S, Robert V, Spouge J L, Levesque C A, Chen W. Fungal barcoding consortium. 2012. Nuclear ribosomal internal transcribed spacer (ITS) region as a universal DNA barcode marker for fungi. Proceedings of the National Academy of Sciences of the United States of America, 109: 6241-6246.
[27] Thomsen M C F, Hasman H, Westh H, Kaya H, Lund O. 2017. RUCS: rapid identification of PCR primers for unique core sequences. Bioinformatics 33 3917-3921.
[28] Wang J, Jacobs J L, Byrne J M, Chilvers M I. 2015. Improved diagnoses and quantification of Fusarium virguliforme, causal agent of soybean sudden death syndrome. Phytopathology 105: 378-387.
[29] Wang M, Lemos B. 2017. Ribosomal DNA copy number amplification and loss in human cancers is linked to tumor genetic context, nucleolus activity, and proliferation. PLoS Genetics 13: e1006994.
[30] Wickes B L, Wiederhold N P. 2018. Molecular diagnostics in medical mycology. Nature communications 9: 5135.
[31] Wu C, Kim Y-S, Smith K M, Li W, Hood H M, Staben C, Selker E U, Sachs M S, Farmant M L. 2009. Characterization of chromosome ends in the filamentous fungus Neurospora crassa. Genetics 181: 1129-1145.
[32] Zhang Z, Schwartz S, Lukas Wagner L, Miller W. 2000. A greedy algorithm for aligning DNA sequences. Journal of Computational Biology 7: 203-214.

The invention claimed is:

1. A method of finding structural variants for identifying and differentiating species, strains and cells in normal and pathological conditions, comprising:
(a) a data storage element storing two or more whole-genome datasets of sequence reads with no genomic location information, where the datasets come from different species or strains, or from cells in normal and pathological conditions; and
(b) a processing element associated with the storage element and configured to:
i. generate, from each dataset of reads, a set of all fragments each associated with its positive number of occurrences (frequency) in the reads in forward and reverse orientation, and arrange the fragments in non-increasing order of their frequency counts, where the length of fragments is less than the length of reads;
ii. store each set of fragments with their frequency counts in non-increasing order of these counts in an output storage element;
iii. obtain every input subset of fragments with their frequency counts≥c (a count cutoff);
iv. generate, from each input subset of fragments, an output subset of fragments such that the frequency counts of all fragments with no matches to fragments in any other subset are greater than f*c, and the frequency counts of all fragments with matches to fragments in other subsets are f times greater than those of the fragments in the other subsets, where the number f is a count factor parameter; and
v. store each output subset of fragments with their frequency counts in non-increasing order in a second output storage element.

2. The method of claim 1, wherein the datasets come from different species or strains of bacteria.

3. The method of claim 1, wherein the datasets come from different species or strains of algae.

4. The method of claim 1, wherein the datasets come from different species or strains of protists.

5. The method of claim 1, wherein the datasets come from different species or strains of fungi.

6. The method of claim 1, wherein the datasets come from different species or strains of plants.

7. The method of claim 1, wherein the datasets come from different species or strains of animals.

8. The method of claim 1, wherein the datasets come from human cells in normal and pathological conditions.

9. The method of claim 1, wherein the datasets come from animal cells in normal and pathological conditions.
